(19) United States
(12) Patent Application Publication
(10) Pub. No.: US 2009/0117230 A1
(43) Pub. Date: May 7, 2009

(54) Phytase-Containing Enzyme Granulate ll

(75) Inventors: Markus Lohscheidt, Heidelberg (DE); Roland Betz, Niederkirchen (DE); Jorg Braun, Essingen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 11/991,833

(22) PCT Filed: Sep. 11, 2006
(86) PCT No.: PCT/EP2006/066220

§ 371 Date: Mar. 11, 2008

(30) Foreign Application Priority Data
Sep. 12, 2005 (DE) 10 2005 043 324.3

(51) Int. Cl. A23K 1/00; C12N 9/14

(52) U.S. Cl. 426/53; 435/195; 426/61

(57) ABSTRACT

The present invention relates to novel phytase-comprising enzyme granules, the particles of which have a weight-average particle size in the range from 300 to 800 μm and the specific phytase activity of which, expressed in FTU/g, is at least 13 000 and does not exceed a value of

FTUₘₐₓ=6000[FTU g⁻¹]·D⁻³·mm³,

D being the weight-average particle diameter of the granule particles in mm.

The invention further relates to phytase-comprising enzyme granules for feeds, the particles of which have a weight-average particle size in the range from 300 to 800 μm and the specific phytase activity of which, expressed in FTU/g, is at least 7000, and does not exceed a value of

FTUₘₐₓ=2000[FTU g⁻¹]·D⁻³·mm³,

D being the weight-average particle diameter of the granule particles in mm.

The invention also relates to the use of the phytase-comprising enzyme granules in feed compositions and, in particular, pelleted feed compositions, which are obtainable using the phytase-comprising enzyme granules.

The present invention relates to novel phytase-comprising enzyme granules which are suitable as feed additives. The invention also relates to the use of the phytase-comprising enzyme granules in feed compositions and, in particular, pelleted feed compositions, which are obtainable using the phytase-comprising enzyme granules.

It is generally customary to add phytase to animal feed in order to ensure better feed utilization, better product quality or lower pollution of the environment. In addition, it is current practice to supply animal feeds in pelleted form, since pelleting not only facilitates feed intake, but also improves handling of the feedstuff. In addition, it has been found that in the case of pelleted feedstuff, certain feed components are digested better, and ingredients added to the feedstuff such as, for example, vitamins, enzymes, trace elements, can be better incorporated in the feed mixture.

To reduce the microbial loading (sanitation) of such animal feeds, heat treating is frequently carried out. A heat treatment also proceeds in the context of the conditioning required for pelleting, in which the feedstuff is admixed with steam and thereby heated and moistened. In the actual pelleting step, the feedstuff is forced through a matrix. Other processes used in the feed industry are extrusion and expansion. The action of heat in all of these processes is a problem, since the enzymes such as phytase present in such feed mixtures are generally thermally unstable. Therefore, various efforts have been made to improve the thermal stability and, in particular, the pelleting stability of enzyme-comprising feed compositions.

With respect to high pelleting stability, it is fundamentally advantageous to produce enzyme granules having comparatively large granule particles, since in these the specific surface area is lower compared with smaller particles. At a high specific enzyme activity, for example at phytase activities greater than 10 000 FTU/g, the metering accuracy with large particles obviously decreases, since uniform distribution of the granules in the feed is difficult to achieve at a preferentially sought activity in the feed of about 500 FTU/kg. The consequence is large variation of the enzyme activity in the daily ration of the animals.

On the other hand, high specific phytase activities of at least 7000 FTU/g or even at least 13 000 FTU/g are desirable for decreasing the manufacturing costs (based on activity) and for reducing the activity-specific product volume.

WO 98/54980 describes enzyme-comprising granules having improved pelleting stability which are produced by extrusion of an aqueous enzyme solution together with a carrier based on an edible carbohydrate and subsequent drying. The phytase activity of the granules is in the range from 5000 to 10 000 FTU/g.

WO 2000/47060 describes phytase-comprising enzyme granules which are suitable as feed additives and which have a polyethylene glycol coating. The specific phytase activity in these particles is in the range from 4000 to 20 000 FTU/g. An association between particle size and phytase activity is not taught. Similar products are disclosed by WO 03/059086 which differ from those of WO 2000/47060 by a coating with a hydrophobic substance instead of the polyethylene glycol coating.

WO 2001/25412 teaches that thick coating layers for enzyme granules (diameter of the entire particle to diameter of enzyme core >1.1) contribute to reduction of the dusting tendency of the product and to increasing storage stability. These thick layers obviously lead to an enlargement of the enzyme granule. Phytase-comprising granules are not taught.

WO 2004/108911 describes enzyme granules having high specific enzyme activity, in particular having a high ratio of active to inactivated enzyme, and to a low dust fraction. The specific phytase activity is at least 15 000 FTU/g. The granules described there are not satisfactory with respect to their metering accuracy.

It is therefore an object of the present invention to provide phytase-comprising enzyme granules having improved metering accuracy with simultaneously high specific phytase activity of at least 13 000 FTU/g. A further object is to provide phytase-comprising enzyme granules having improved metering accuracy with simultaneously specific phytase activity of 7000 to 13 000 FTU/g. The granules should, in addition, have high pelleting stability and be able to be produced in a simple manner and inexpensively. In addition, no losses in enzyme activity should occur even during the production.

It has surprisingly been found that phytase-comprising enzyme granules at a high specific phytase activity of at least 13 000 FTU/g, preferably at least 14 000 FTU/g, in particular at least 15 000 FTU/g, particularly preferably at least 16 000 FTU/g, very particularly preferably at least 20 000 FTU/g, exhibit particularly good meterability when the mean particle size (weight average) of the granule particles is in the range from 300 to 800 μm, preferably in the range from 320 to 700 μm, in particular in the range from 350 to 600 μm, and especially in the range from 400 to 550 μm, and the specific phytase activity of the granules, expressed in FTU units per gram, does not exceed a value of 6000 [FTU g⁻¹]˜D⁻³·mm³, preferably 5000 [FTU g⁻¹]·D⁻³ mm³, in particular 4000 [FTU g⁻¹]·D⁻³·mm³, particularly preferably 3000 [FTU g⁻¹]·D⁻³·mm³, and very particularly preferably 2000 [FTU g⁻¹]·D⁻³·mm³. In other words, the maximum phytase activity FTUₘₐₓ which the granules may have is correlated with the weight-average particle diameter of the granule particles according to the following formula:

FTUₘₐₓ=6000[FTU g⁻¹]·D⁻³·mm³,

where D is the weight-average particle diameter of the granule particles in mm.

It has in addition been found that phytase-comprising enzyme granules having a specific phytase activity of at least 7000 FTU/g, preferably at least 7500 FTU/g, in particular at least 8000 FTU/g, for example 7000 to 13 000 FTU/g, or 7500 to 13 000 FTU/g, or 8000 to 13 000 FTU/g exhibit particularly good meterability when the mean particle size (weight average) of the granule particles is in the range from 300 to 800 μm, preferably in the range from 320 to 700 μm, in particular in the range from 350 to 600 μm, and especially in the range from 400 to 550 μm and the specific phytase activity of the granules, expressed in FTU units per gram, does not exceed a value of 2000 [FTU g⁻¹]·D⁻³·mm³, particularly preferably 1500 [FTU g⁻¹]·D⁻³·mm³.

Accordingly, the invention firstly relates to phytase-comprising enzyme granules for feeds, the particles of which have a weight-average particle size in the range from 300 to 800 μm and the specific phytase activity of which, expressed in FTU/g, is at least 13 000, and does not exceed a value of

FTUₘₐₓ=6000[FTU g⁻¹]·D⁻³·mm³,

D being the weight-average particle diameter of the granule particles in mm.

The specific phytase activity of such inventive enzyme granules is preferably at least

13 000 FTU/g, in particular at least 14 000 FTU/g, particularly preferably at least
15 000 FTU/g, very particularly preferably at least 16 000 FTU/g, and especially

### 20 000 FTU/g.

Preferably, the specific phytase activity will not exceed a value of

FTUₘₐₓ=5000 [FTU g⁻¹]·D⁻³·mm³, in particular of
FTUₘₐₓ=4000 [FTU g⁻¹]·D⁻³·mm³, particularly preferably of
FTUₘₐₓ=3000 [FTU g⁻¹]·D⁻³·mm³, and in particular preferably of
FTUₘₐₓ=2000 [FTU g⁻¹]·D⁻³·mm³.

The invention further relates to a phytase-comprising enzyme granule for feeds, the particles of which have a weight-average particle size in the range from 300 to 800 μm and the specific phytase activity of which, expressed in FTU/g, is at least 7000 and does not exceed a value of

FTUₘₐₓ=2000[FTU g⁻¹]·D⁻³·mm³,

D being the weight-average particle diameter of the granule particles in mm.

The specific phytase activity of such inventive enzyme granules is preferably at least 7500 FTU/g, in particular at least 8000 FTU/g.

Preferably, the specific phytase activity will not exceed a value of FTUₘₐₓ=1500 [FTU g⁻¹]·D³·mm³, in particular of 1000 [FTU g⁻¹]·D⁻³·mm³.

1 FTU of phytase activity is defined in this case as the amount of enzyme which releases 1 micromol of inorganic phosphate per minute from 0.0051 mol/l aqueous sodium phytate at pH 5.5 and 37° C. The phytase activity can be determined, for example, according to “Determination of Phytase Activity in Feed by a Colorimetric Enzymatic Method”: Collaborative Interlaboratory Study Engelen et al.: Journal of AOAC International Vol. 84, No. 3, 2001, or else Simple and Rapid Determination of Phytase Activity, Engelen et al., Journal of AOAC International, Vol. 77, No. 3, 1994.

The weight-average particle diameter of the granule particles is preferably in the range from 320 to 700 μm, in particular in the range from 350 to 600 μm, and especially in the range from 400 to 550 μm. The mean particle size distribution can be determined in a manner known per se by light scattering, for example using a Mastersizer S, from Malvern Instruments GmbH or by sieve analysis, for example using a sieve machine Vibro VS 10000 from Retsch. Those skilled in the art will take mean particle size to mean the D₅₀ value of the particle size distribution curve, that is the value which 50% by weight of all particles exceed or undershoot.

Preferably, the particles have only a small fraction of finely divided particles. Accordingly, at least 90% by weight of the particles have a particle diameter of greater than 100 μm, in particular greater than 150 μm, and especially greater than 200 μm, and no more than 10% by weight have a diameter below these limits (D₁₀ value). Preferably, the weight fraction of particles having a particle diameter less than 100 μm is less than 8% by weight, in particular less than 5% by weight, and especially less than 1% by weight.

Preferably, the granule particles have only a small coarse particle fraction. Accordingly, at least 90% by weight of the particles have a particle diameter of no greater than 1200 μm, in particular no greater than 1000 μm, and especially no greater than 800 μm, and no more than 10% by weight have a diameter above these limits (D₁₀ value). Preferably, the weight fraction of particles having a particle diameter of 1200 μm, in particular above 1000 μm is less than 8% by weight, in particular less than 5% by weight, and especially less than 1% by weight.

Preferably, the particle size distribution is narrow, that is the deviation from the weight-average diameter is small. In preferred granules, therefore, the particle size distribution is characterized by a D₉₀/D₁₀ value <3.5, in particular <3, particularly preferably <2, and especially <1.8.

The granule particles can in principle have an irregular geometry, particles having a regular, that is cylindrical or spherical geometry, being preferred. In particular, it has proved to be expedient when the granule particles have a roundness factor <2, in particular <1.8, and especially <1.6. The roundness factor corresponds to the ratio of the median surface area of the granule particles to the surface area of a sphere which has the weight-average diameter of the granule particles.

In a first preferred embodiment, the granule particles have a spherical or ellipsoidal geometry, the ratio of the largest to smallest diameter preferably not exceeding a value of 3:1, in particular 1.5:1, and especially 1.3:1. In an equally preferred embodiment, the geometry of the granule particles is cylindrical, the ratio of diameter to length preferably being in the range from 1:1.3 to 1:3. The cylindrical granule particles preferably have rounded ends.

The inventive granules can be uncoated, or the granule particles have a coating, that is the granule particles comprise an enzyme-comprising core A and a coating B arranged on the core.

The inventive granule particles comprise, in addition to the phytase, preferably at least one solid carrier material suitable for feeds. In coated granules, the carrier material is typically a component of the core. The carrier material typically makes up at least 50% by weight, in particular at least 55% by weight, and frequently at least 60% by weight, of the nonaqueous components of the uncoated granule or of the core, for example 50 to 96.9% by weight, preferably 55 to 94.8% by weight, and in particular 60 to 89.7% by weight.

As feed-compatible carrier materials, use can be made of customary inert inorganic or organic carriers. An “inert” carrier must not exhibit any adverse interactions with the enzyme(s) of the inventive feed additive, such as, for example, cause irreversible inhibition of the enzyme activity, and must be harmless for use as an auxiliary in feed additives. Examples of suitable carrier materials which may be mentioned are: low-molecular-weight organic compounds, and also higher-molecular-weight organic compounds of natural or synthetic origin, and also inert inorganic salts. Preference is given to organic carrier materials. Among these, carbohydrates are particularly preferred.

Examples of suitable low-molecular-weight organic carriers are, in particular, sugars such as, for example, glucose, fructose, sucrose. Examples of higher-molecular-weight organic carriers which may be mentioned are carbohydrate polymers, in particular those which comprise α-D-glucopyranose, amylose or amylopectin units, in particular native and modified starches, microcrystalline cellulose, but also α-glucans and β-glucans, pectin (including protopectin) and glycogen. Preferably, the carrier material comprises at least one water-insoluble polymeric carbohydrate, in particular a native starch material such as, in particular, corn starch, rice starch, wheat starch, potato starch, starches of other plant sources such as starch from tapioca, cassaya, sago, rye, oats, barley, sweet potatoes, arrowroot and the like, in addition cereal flours such as, for example, corn flour, wheat flour, rye flour, barley flour and oat flour, and also rice flour. Suitable materials are, in particular, also mixtures of the abovementioned carrier materials, in particular mixtures which predominantly, i.e. at least 50% by weight, based on the carrier material, comprise one or more starch materials. Preferably, the water-insoluble carbohydrate makes up at least 50% by weight, in particular at least 65% by weight, and especially at least 80% by weight, of the carrier material. Particularly preferred carrier materials are starches which comprise no more than 5% by weight, and in particular no more than 2% by weight, of protein or other components. A further preferred carrier material is microcrystalline cellulose. This can be used alone or in a mixture with the abovementioned carrier materials. If the microcrystalline cellulose is used in a mixture with other carrier materials, it preferably makes up no more than 50% by weight, in particular no more than 30% by weight, for example 1 to 50% by weight, in particular 1 to 30% by weight, and especially 1 to 10% by weight, of the carrier material.

Inorganic carrier materials which come into consideration are in principle all inorganic carrier materials known for feeds and feed additives, for example inert inorganic salts, for example sulfates or carbonates of alkali and alkaline earth metals such as sodium, magnesium, calcium and potassium sulfate or carbonate, in addition feed-compatible silicates such as talcum and silicic acids. The amount of inorganic carrier material, based on the total amount of carrier material, will generally not exceed 50% by weight, particularly 35% by weight, and very particularly 20% by weight. In a preferred embodiment, the organic carrier materials make up the total amount or virtually the total amount, that is at least 95% by weight, of the carrier material.

In addition, the granules comprise at least one phytase, mixtures of different phytases or mixtures of phytase with one or more other enzymes also being able to be present. Typical enzymes for feeds are, in addition to phytase, for example oxidoreductases, transferases, lyases, isomerases, ligases, lipases, and hydrolases different from phytase. In coated granules, the phytase is typically a component of the core. Typical enzymes for feeds are, in addition to phytase, for example oxidoreductases, transferases, lyases, isomerases, ligases, lipases, and in particular hydrolases different from phytase. Examples of hydrolases, that is enzymes which cause a hydrolytic cleavage of chemical bonds, are esterases, glycosidases, keratinases, ether hydrolases, proteases, amidases, aminidases, nitrilases, and phosphatases. Glycosidases (EC 3.2.1, also termed carbohydrases) comprise not only endo- but also exoglycosidases, which cleave not only α- but also β-glycosidic bonds. Typical examples thereof are amylases, maltases, cellulases, endoxylanases, for example endo-1,4-β-xylanase or xylan endo-1,3-β-xylosidase, β-glucanases, in particular endo-1,4-β- and endo-1,3-β-glucanases, mannanases, lysozymes, galactosidases, pectinases, β-glucuronidases and the like.

The expression “phytase” comprises not only natural phytase, but also any other enzyme which exhibits phytase activity, for example is capable of catalyzing a reaction which liberates the phosphorus or phosphate from myoinositol phosphates. The phytase can be not only a 3-phytase (EC 3.1.3.8) but also a 4- or 6-phytase (EC 3.1.3.26) or a 5-phytase (EC 3.1.3.72) or a mixture thereof. Preferably, the phytase belongs to the enzyme class EC 3.1.3.8.

The phytase used according to the invention is not subject to any restrictions and can be not only of microbiological origin, but also a phytase obtained by genetic modification of a naturally occurring phytase, or by de-novo construction. The phytase can be a phytase from plants, from fungi, from bacteria, or a phytase produced by yeasts. Preference is given to phytases from microbiological sources such as bacteria, yeasts or fungi. However, they can also be of plant origin. In a preferred embodiment, the phytase is a phytase from a fungal strain, in particular from an Aspergillus strain, for example Aspergillus niger, Aspergillus oryzae, Aspergillus ficuum, Aspergillus awamori, Aspergillus fumigatus, Aspergillus nidulans or Aspergillus terreus. Particular preference is given to phytases which are derived from a strain of Aspergillus niger or a strain of Aspergillus oryzae. In another preferred embodiment, the phytase is derived from a bacterial strain, in particular a Bacillus strain, an E. coli strain or a Pseudomonas strain, among these phytases being preferred which are derived from a Bacillus subtilis strain. In another preferred embodiment, the phytase is derived from a yeast, in particular a Kluveromyces strain or a Saccharomyces strain, among these phytases being preferred which are derived from a strain of Saccharomyces cerevisiae. In this invention, the expression “an enzyme derived from phytase” comprises the phytase naturally produced by the respective strain which is either obtained from the strain, or that is coded for by a DNA sequence isolated from the strain and is produced by a host organism which has been transformed using this DNA sequence. The phytase can be obtained from the respective microorganism by known techniques which typically comprise fermentation of the phytase-producing microorganism in a suitable nutrient medium (see, for example, ATCC catalog) and subsequently obtaining the phytase from the fermentation medium by standard techniques. Examples of phytases and of methods for preparing and isolating phytases may be found in EP-A 420358, EP-A 684313, EP-A 897010, EP-A 897985, EP-A 10420358, WO 94/03072, WO 98/54980, WO 98/55599, WO 99/49022, WO 00/43503, WO 03/102174, the contents of which are hereby explicitly incorporated by reference.

The amount of phytase in the granules obviously depends on the desired activity of the enzyme granules and the activity of the enzyme used and is typically in the range from 3 to 49.9% by weight, in particular in the range from 5 to 44.8% by weight, and especially in the range from 10 to 39.7% by weight, calculated as dry mass and based on the total weight of all nonaqueous components of the uncoated granules or the core.

In a preferred embodiment, the components of the uncoated granules or the core, in addition to the feed-compatible carrier material, comprise at least one water-soluble polymer. This polymer acts as binder and at the same time increases the pelleting stability. Preferred water-soluble polymers exhibit a number-average molecular weight in the range from 5×10³ to 5×10⁶ dalton, in particular in the range from 1×10⁴ to 1×10⁶ dalton. The polymers are water-soluble when at least 3 g of polymer may be dissolved completely in 1 liter of water.

The water-soluble polymers used according to the invention comprise
polysaccharides, for example water-soluble modified starches generally having adhesive properties, for example starch breakdown products (dextrins) such as acid dextrins, pyrodextrins, enzymatic partial hydrolysates (limited dextrins), oxidatively broken down starches and their reaction products of dextrins with cationic or anionic polymers, reaction products of dextrins with octenyl succinate anhydride (OSA), starch-based adhesive, in addition chitin, chitosan, carragheen, alginates, arabic acid salts, gums, e.g. gum Arabic, tragacanth, karaya gum; xanthan gum and gellan gum; galactomannans; water-soluble cellulose derivatives, for example methylcellulose, ethylcellulose and hydroxyalkylcelluloses such as, for example, hydroxyethylcellulose (HEC), hydroxyethyl methylcellulose (HEMC), ethyl hydroxyethylcellulose (EHEC), hydroxypropylcellulose (HPC), hydroxypropyl methylcellulose (HPMC) and hydroxybutylcellulose, and also carboxymethylcellulose (CMC);
water-soluble proteins, e.g. proteins of animal origin such as gelatin, casein, in particular sodium caseinate and plant proteins such as soy protein, pea protein, bean protein, rapeseed protein, sunflower protein, cottonseed protein, potato protein, lupin, zein, wheat protein, corn protein and rice protein,
synthetic polymers, for example polyethylene glycol, polyvinyl alcohol and, in particular, the kollidon brands of BASF, vinyl alcohol/vinyl ester copolymers, homo- and copolymers of vinylpyrrolidone with vinyl acetate and/or C₁-C₄-alkyl acrylates,
and biopolymers modified if appropriate, e.g. lignin, polylactide.

Preferred water-soluble polymers are neutral, that is they have no acidic or basic groups. Among these, polyvinyl alcohols, including partially saponified polyvinyl acetates having a degree of saponification of at least 80%, and also, in particular, water-soluble, neutral cellulose ethers such as methylcellulose, ethylcellulose and hydroxyalkylcelluloses such as, for example, hydroxyethylcellulose (HEC), hydroxyethyl methylcellulose (HEMC), ethyl hydroxyethylcellulose (EHEC), hydroxypropylcellulose (HPC), hydroxypropyl methylcellulose (HPMC) and hydroxybutylcellulose are particularly preferred.

In a preferred embodiment of the invention, the water-soluble polymer is selected from neutral cellulose ethers. Examples of inventively preferred water-soluble neutral cellulose ethers are methylcellulose, ethylcellulose and hydroxyalkylcelluloses, for example hydroxyethylcellulose (HEC), hydroxyethyl methylcellulose (HEMC), ethyl hydroxyethylcellulose (EHEC), hydroxypropylcellulose (HPC), hydroxypropyl methylcellulose (HPMC) and hydroxybutylcellulose. Among these, methylcellulose, ethylcellulose and mixed cellulose ethers having methyl groups or ethyl groups and hydroxyalkyl groups such as HEMC, EHEC and HPMC are particularly preferred. Preferred methyl- or ethyl-substituted cellulose ethers have a degree of substitution DS (with respect to the alkyl groups) in the range from 0.8 to 2.2 and, in the case of mixed cellulose ethers, a degree of substitution DS with respect to the alkyl groups in the range from 0.5 to 2.0, and a degree of substitution HS with respect to the hydroxyalkyl groups in the range from 0.02 to 1.0.

The fraction of water-soluble polymers can be varied over wide ranges, depending on the embodiment. In the case of enzyme granules having a carrier component, their fraction makes up preferably 0.1 to 20% by weight, in particular 0.2 to 10% by weight, and especially 0.3 to 5% by weight of the non-aqueous components of the uncoated enzyme granule or of the core.

In addition, the core-forming or the uncoated granule-forming material can additionally comprise a salt stabilizing the enzyme. Stabilizing salts are typically salts of divalent cations, in particular salts of calcium, magnesium or zinc, and also salts of monovalent cations, in particular sodium or potassium, for example the sulfates, carbonates, hydrogencarbonates and phosphates including hydrogenphosphates and ammonium hydrogenphosphates of these metals. Preferred salts are sulfates. Particular preference is given to magnesium sulfate and zinc sulfate, including their hydrates. The amount of salt is preferably in the range from 0.1 to 10% by weight, in particular in the range from 0.2 to 5% by weight, and especially in the range from 0.3 to 3% by weight, based on the total weight of all nonaqueous components of the core material or the uncoated granules.

The inventive granules generally have a water content below 15% by weight, frequently in the range from 1 to 12% by weight, in particular in the range from 3 to 10% by weight, and especially in the range from 5 to 9% by weight, based on the weight of the enzyme-comprising granules.

According to a preferred embodiment of the invention, the granule particles of the enzyme granules comprise
a) a core which comprises at least one phytase and at least one solid carrier material suitable for feeds, and
b) a coating arranged on the core.

With respect to the pelleting stability, it has proved advantageous when the uncoated granules, or the cores of the coated granules, or the entire particle after, if appropriate, required grinding, on suspension or dissolution in demineralized water at 25° C. give a pH in the range of 4.5 to 6.5, preferably 4.6 to 6, and particularly preferably of 4.7 to 5.5. Generally, to determine the pH, 5 g of the uncoated cores or coated cores are dissolved at 25° C. in 200 ml of demineralized water and the pH established after 30 min is determined using a glass electrode or a pH measuring instrument.

According to a preferred embodiment of the invention, the core-forming or the uncoated granule-forming components accordingly, in addition to the phytase and also the further components present if appropriate such as solid carrier material and binder, comprise at least one agent for setting a pH of 4.5 to 6.5, preferably 4.6 to 6.0, and particularly preferably of 4.7 to 5.5, for example a buffer or a base, the latter, in particular, when the core-forming materials themselves have acid groups.

Suitable substances for setting the pH are sufficiently known to those skilled in the art, for example from Küster-Thiel, Rechentafeln für die chemische Analytik [Calculation tables for chemical analysis], 102nd edition, 1982, Walter de Gruyter-Verlag and Handbook of Chemistry and Physics, 76th ed. 1995-1996, CRS Press 8-38 ff.; DIN Normenheft 22, Richtlinien für die pH-Messung in industriellen Anlagen [Guidelines for pH measurement in industrial plants], Berlin: Beuth 1974; DIN 19266 (August 1979); DIN 19267 (Aug. 1978); Naturwissenschaften 65, 438 ff. (1978). Kontakte (Merck) 1981, No. 1, 37-43.

Examples of suitable buffers are acetate, propionate, tartrate, hydrogencarbonate, phthalate, hydrogenphthalate, in particular the sodium, potassium or calcium salts of the abovementioned substances, including their hydrates or dihydrates, phosphate buffer, potassium or sodium phosphate, their hydrates or dihydrates, sodium or potassium carbonate. Examples of suitable bases are sodium or potassium carbonate, sodium, potassium, calcium, magnesium, ammonium hydroxide, or ammonia water or oxides thereof.

The amount of buffer or base is typically in the range from 0.1 to 5% by weight, based on the total weight of the core-forming or the uncoated granule-forming nonaqueous components. In principle there need be no addition of buffer when the components of the core material in the composition present in the core already give such a pH. In particular, granules have proven useful which are obtainable by a method in which, to produce the granules, use is made of an aqueous enzyme concentrate which, at 25° C., has a pH in the range from 4.5 to 6.5, preferably 4.6 to 6, and particularly preferably from 4.7 to 5.5. In this case the pH of the enzyme concentrate is determined directly using a glass electrode or a pH measuring instrument.

If the particles of the inventive enzyme granules have at least one coating arranged on the core of the particles, this coating covers preferably at least 80% (mean value) of the surface area of the cores, and in particular covers the cores completely.

The weight ratio of core to coating is preferably in the range from 70:30 to 99:1, preferably in the range from 75:25 to 98:2, in particular in the range from 80:20 to 96:4, and especially in the range from 85:15 to 95:5; in some cases, higher coating fractions can also be advantageous, for example in the case of salt coatings.

Typically, the particle sizes of the inventive coated enzyme granules correspond to those of the uncoated granules or of the cores, which are also termed raw granules hereinafter. In other words, in coated granules the ratio of median particle diameter of the coated granules to the median particle diameter of the raw granules will generally not exceed a value of 1.1:1, and in particular a value of 1.09:1.

Suitable coatings are in principle all types of coatings which are known for enzyme granules from the prior art. Preference is given to hydrophobic coatings, that is coatings whose components are water-insoluble or of only limited water solubility. Accordingly, an inventively particularly preferred embodiment relates to phytase-comprising enzyme granules whose particles have a coating at least 90% by weight of which comprises water-insoluble hydrophobic substances.

Hydrophobic materials which come into consideration for the hydrophobic coating are not only polymeric substances but also oligomeric or low-molecular-weight substances. According to the invention, the hydrophobic materials have a high hydrocarbon fraction, the fraction of carbon and hydrogen generally making up at least 80% by weight, in particular at least 85% by weight, of the hydrophobic material. Preference is given to those substances which have a melting point above 30° C., more preferably above 40° C., in particular above 45° C., and especially above 50° C., or in the case of non-melting substances are solid at these temperatures or have a glass transition temperature above these temperatures. Preference is given to hydrophobic materials having melting points in the range from 40 to 95° C., in particular in the range from 45 to 80° C., and particularly preferably in the range from 50 to 70° C.

Preferably, the hydrophobic material is low-acid, and has an acid value less than 80, in particular less than 30, and especially less than 10 (determined as specified in ISO 660).

Examples of hydrophobic materials suitable according to the invention are
polyolefins such as polyethylene, polypropylene and polybutenes;
saturated fatty acids preferably having 10 to 32 carbon atoms, frequently 12 to 24 carbon atoms, and in particular 16 to 22 carbon atoms;
esters of saturated fatty acids, preferably mono-, di- and triglycerides and also esters of saturated fatty acids with fatty alcohols. The fatty alcohols for example have 10 to 32 carbon atoms, in particular 16 to 24 carbon atoms, such as cetyl alcohol or stearyl alcohol. The fatty acids and the fatty acid radicals in the esters of fatty acids preferably have 10 to 32, frequently 12 to 24, carbon atoms and, in particular, 16 to 22 carbon atoms;
waxes, in particular plant waxes and waxes of animal origin, but also montan waxes and montan ester waxes;
polyvinyl acetates;
C₁-C₁₀-alkyl (meth)acrylate polymers and copolymers, preferably those having a number-average molecular weight of from about 100 000 to 1 000 000; in particular ethyl acrylate/methyl methacrylate copolymers and methyl acrylate/ethyl acrylate copolymers.

In a preferred embodiment, the coating-forming material comprises up to at least 70% by weight, particularly up to at least 80% by weight, in particular up to at least 90% by weight, of at least one substance selected from saturated fatty acids, esters of fatty acids and mixtures thereof (called “fats” below for short), esters of fatty acids and, in particular, triglycerides being preferred. Saturated means that the hydrophobic material is essentially free from unsaturated components and correspondingly has an iodine value less than 5 and, in particular, less than 2 (method according to Wijs, DIN 53 241). Particularly preferably, the coating comprises up to at least 70% by weight, in particular at least 80% by weight, and especially at least 90% by weight, of the above-mentioned triglycerides.

In a preferred embodiment of the invention, the coating agent predominantly, that is up to at least 70% by weight, in particular at least 80% by weight, and especially greater than 90% by weight, comprises hydrogenated vegetable oils, in particular triglycerides of plant origin, for example hydrogenated cottonseed, corn, peanut, soybean, palm, palm kernel, babassu, rapeseed, sunflower and safflower oils. Hydrogenated vegetable oils which are particularly preferred among these are hydrogenated palm oil, cottonseed oil and soybean oil. The most preferred hydrogenated vegetable oil is hydrogenated soybean oil. Similarly, other fats and waxes originating from plants and animals are also suitable, for example beef tallow. Suitable materials are also nature-identical fats and waxes, that is synthetic waxes and fats having a composition which predominantly corresponds to that of the natural products.

The table below mentions some examples of coating materials which are suitable according to the invention:

| | | Melting | |
|---|---|---|---|
| Name | Composition | range | CAS No./INCI |
| Cutina CP from | synthetic cetyl palmitate | 46-51° C. | 95912-87-1 |
| Cognis | | | cetyl palmitate |
| Edenor NHTI-G | Triglyceride | 56-60° C. | 67701-27-3* |
| from Cognis | | | |
| Edenor NHTI-V from | Triglyceride | 57-60° C. | 67701-27-3* |
| Cognis | | | EINECS 266-945-8 |
| Edenor C1892 from | Stearic acid, C16-18 | 66-99° C. | |
| Cognis | | | |
| Edenor HPA from | Fatty acids, palm oil, | 55-57° C. | |
| Cognis | hydrogenated, C16-18 | | |
| Edenor HRAGW | Fatty acids, C16-22 | 64-66° C. | |
| from Cognis | | | |
| Edenor C2285R | Fatty acids, C18-22 | 75-78° C. | 68002-88-0* |
| from Cognis | | | |
| Rilanit from Cognis | Triglyceride | 83-90° C. | |
| Japan wax | principally glycerol palmitate | 49-55° C. | rhus succedanea |
| substitute from Kahl- | | | |
| Wachsraffinerie | | | |
| Tefacid from | palm kernel oil Tefacid Palmic 90 | 65° C. | 57-10-3 |
| Karlshamns | | | |
| Soybean fat powder | | 65-70° C. | |
| from Sankyu/Japan | | | |

Suitable fats are also the products of the company Aarhus Olie, Denmark, marketed under the trademark Vegeol PR, for example Vegeol® PR 267, PR 272, PR 273, PR 274, PR 275, PR 276, PR 277, PR 278 and PR 279.

Waxes suitable as coating materials are, in particular, waxes of animal origin such as beeswax and lanolin, waxes of plant origin such as candelilla wax, carnauba wax, cane sugar wax, caranday wax, raffia wax, Columbia wax, esparto wax, alfalfa wax, bamboo wax, hemp wax, Douglas fir wax, cork wax, sisal wax, flax wax, cotton wax, dammar wax, cereal wax, rice wax, ocatilla wax, oleander wax, montan waxes, montan ester waxes, polyethylene waxes, in addition the products of Süddeutsche Emulsions-Chemie marketed under the trademarks Wükonil, Südranol, Lubranil or Mikronil, or the BASF products having the trademarks Poligen WE1, WE3, WE4, WE6, WE7, WE8 BW, WE9.

Suitable hydrophobic coating materials are, in addition, the following polyolefins: polyisoprene, medium- and high-molecular-weight polyisobutene and polybutadiene.

In preferred alkyl, (meth)acrylate polymers and copolymers, the alkyl group has 1 to 4 carbon atoms. As specific examples of suitable copolymers, mention may be made of: ethyl acrylate/methyl methacrylate copolymers, which are marketed, for example, under the trademarks Kollicoat EMM 30D by BASF AG, or under the trademark Eudragit NE 30 D by Degussa; and also methacrylate/ethyl acrylate copolymers as are marketed, for example, under the trademark Kollicoat MAE 30DP by BASF AG, or under the trademark Eudragit 30/55 by Degussa in the form of an aqueous dispersion.

Examples of polyvinyl acetate dispersions which may be mentioned are those which are stabilized by polyvinylpyrrolidone and are marketed, for example, under the trademark Kollicoat SR 30D by BASF AG (solids content of the dispersion about 20 to 30% by weight).

In another embodiment of the invention, the coating comprises polymeric substances which have in water an at least limited solubility. Examples of these are
a) polyalkylene glycols, in particular polyethylene glycols, preferably those having a number-average molecular weight of from about 400 to 15 000, such as, for example, about 400 to 10 000;
b) polyalkylene oxide polymers or copolymers, preferably those having a number-average molecular weight of from about 4000 to 20 000, such as, for example, about 7700 to 14 600; in particular block copolymers of polyoxyethylene and polyoxypropylene;
c) polyvinylpyrrolidone, preferably having a number-average molecular weight of from about 7000 to 1 000 000, such as, for example, about 44 000 to 54 000;
d) vinylpyrrolidone/vinyl acetate copolymers, preferably those having a number-average molecular weight of from about 30 000 to 100 000, such as, for example, about 45 000 to 70 000;
e) polyvinyl alcohols, preferably those having a number-average molecular weight of from about 10 000 to 200 000, such as, for example, about 20 000 to 100 000;
f) modified celluloses and cellulose derivatives, such as, for example, hydroxypropyl methylcellulose, preferably having a number-average molecular weight of from about 6000 to 80 000, such as, for example, about 12 000 to 65 000; or else methylcellulose, ethylcellulose and hydroxyalkylcelluloses such as, for example, hydroxyethylcellulose (HEC), hydroxyethyl methylcellulose (HEMC), ethyl hydroxyethylcellulose (EHEC), hydroxypropylcellulose (H PC) and
g) polyvinyl alcohol-polyethylene glycol graft copolymers;
h) modified starches (for example reaction products of octenyl succinate anhydride (OSA) and starch).

Examples of suitable polyalkylene glycols a) which may be mentioned are: polypropylene glycols and, in particular, polyethylene glycols of a different molar mass, such as, for example, PEG 4000 or PEG 6000, obtainable from BASF AG under the trademarks Lutrol® E 4000 and Lutrol® E 6000, and also the Kollidon brands from BASF.

Examples of the above polymers b) which may be mentioned are: polyethylene oxides and polypropylene oxides, ethylene oxide/propylene oxide mixed polymers and also block copolymers, made up from polyethylene oxide and polypropylene oxide blocks, such as, for example, polymers which are obtainable from BASF AG under the trademark Lutrol® F68 and Lutrol® F127.

Examples of the above polymers c) which may be mentioned are: polyvinylpyrrolidones, as are marketed, for example, by BASF AG under the trademark Kollidon® or Luviskol®.

An example of the abovementioned polymers d) which may be mentioned is: a vinylpyrrolidone/vinyl acetate copolymer which is marketed by BASF AG under the trademark Kollidon® VA64.

Examples of the above polymers e) which may be mentioned are: products, as are marketed, for example, by Clariant under the trademark Mowiol®.

Examples of suitable polymers f) which may be mentioned are: hydroxypropyl methylcelluloses, as are marketed, for example, by Shin Etsu under the trademark Pharmacoat®.

Examples of polymers g) are the products of BASF Aktiengesellschaft marketed under the trademark Kollicoat® IR.

Of course, the inventive enzyme granules, in addition to the hydrophobic coating, can also have one or more, for example, 1, 2 or 3, further coatings which comprise other materials, for example the coatings taught in the prior art. It is essential to the invention that at least one coating consists of the hydrophobic materials, this layer being able to be arranged as desired and, in particular, arranged directly on the enzyme-comprising core. There is in addition the possibility that the at least one layer is a salt layer or a layer which comprises at least 30% salt. Such a salt layer will preferably be arranged between the core and the outermost layer. The salts mentioned above can be mentioned here as example.

The inventive enzyme granules can be produced by analogy with known production methods for enzyme granules, for example analogously to the procedures described in WO 98/54980, WO 98/55599, WO 01/00042, WO 03/059086, WO 03/059087, WO 2004/108911 or PCT/EP 2005/000826.

According to a preferred embodiment, the method comprises the following steps:
a) providing uncoated, phytase-comprising raw granules which comprise at least one phytase and at least one solid carrier material suitable for feeds, and which generally have a water content below 15% by weight, frequently in the range from 1 to 12% by weight, in particular in the range from 3 to 10% by weight, and especially in the range from 5 to 9% by weight, based on the weight of the enzyme-comprising raw granules, and
b) coating the raw granules.

The raw granules can be produced in principle in any desired manner. For example, a mixture comprising the feed-compatible carrier, at least one water-soluble, neutral cellulose derivative, and at least one enzyme and if appropriate further components such as water, buffer, stabilizing metal salts, can be processed to form raw granules in a manner known per se by extrusion, mixer-granulation, fluidized-bed granulation, disk agglomeration or compacting. Uncoated granules are produced in a similar manner to the production of raw granules, with the difference that the granules are not coated.

In a preferred embodiment, production of the raw granules comprises in a first step the extrusion of a water-comprising dough which comprises at least one enzyme and at least one inert, preferably solid carrier material and if appropriate further components such as water, buffer, stabilizing metal salts and binders in the amounts stated above.

Preferably, production of the dough comprises setting the pH in such a manner that the dough, on suspension in water, has a pH in the range from 4.5 to 6.5, preferably 4.6 to 6, and particularly preferably from 4.7 to 5.5. The pH can be set by adding a buffer or a base to the dough. Preferably, the pH of the dough is set in such a manner that the dough is produced using an aqueous enzyme concentrate whose pH on dilution is in the range from 4.5 to 6.5, more preferably 4.6 to 6, and particularly preferably from 4.7 to 5.5. Since the enzyme concentrate frequently has a slightly acidic pH below 4, preferably a buffer or a base will be added. Suitable bases are, in addition to ammonia, ammonia water and ammonium hydroxide, hydroxides, citrates, acetates, formates, carbonates and hydrogencarbonates of alkali metals and alkaline earth metals, and also amines and alkaline earth metal oxides such as CaO and MgO. Examples of inorganic buffering agents are alkali metal hydrogenphosphates, in particular sodium and potassium hydrogenphosphates, for example K₂HPO₄, KH₂PO₄ and Na₂HPO₄. A preferred agent for setting the pH is ammonia or ammonia water, NaOH, KOH. Suitable buffers are, for example, mixtures of aforesaid bases with organic acids such as acetic acid, formic acid, citric acid.

The carrier material generally makes up 50 to 96.9% by weight, preferably 55 to 94.8% by weight, and in particular 60 to 89.7% by weight of the nonaqueous components of the dough. The at least one, water-soluble, polymeric binder generally makes up 0.1 to 10% by weight, preferably 0.15 to 5% by weight, in particular 0.2 to 2% by weight, and especially 0.3 to 1% by weight, of the nonaqueous components of the dough. The at least one enzyme generally makes up 3 to 49.9% by weight, in particular in the range from 5 to 44.8% by weight, and especially in the range from 10 to 39.7% by weight, of the nonaqueous components of the dough. The fraction of other components corresponds to the weight fractions given above for the composition of the core or uncoated granules.

In addition to aforesaid components, the dough comprises water in an amount which ensures sufficient homogenization for the dough-forming components and adequate consistency (plasticization) of the dough for extrusion. The amount of water required for this can be determined in a manner known per se by those skilled in the art in the field of enzyme formulation. The water fraction in the dough is typically in the range from >15 to 50% by weight, in particular in the range from 20 to 45% by weight, and especially in the range from 25 to 40% by weight, based on the total weight of the dough.

The dough is produced in a manner known per se by mixing the dough-forming components in a suitable mixing apparatus, for example in a conventional mixer or kneader. For this, the solid or solids, for example the carrier material, are intensively mixed with the liquid phase, for example water, an aqueous binder solution, or an aqueous enzyme concentrate. Generally, the carrier will be introduced as solid into the mixer and mixed with an aqueous enzyme concentrate and also with the water-soluble polymer, preferably in the form of a separate aqueous solution or dissolved in the aqueous enzyme concentrate, and also if appropriate with the stabilizing salt, preferably in the form of a separate aqueous solution or suspension, in particular dissolved or suspended in the aqueous enzyme concentrate. If appropriate, further water will be added to set the desired consistency of the dough. Preferably, during mixing, a temperature of 60° C., in particular 40° C., will not be exceeded. Particularly preferably, the temperature of the dough during mixing is 10 to 30° C. If appropriate, therefore, the mixing apparatus will be cooled during dough production.

The resultant dough is subsequently subjected to an extrusion, preferably an extrusion at low pressure. The extrusion, in particular extrusion at low pressure, generally proceeds in an apparatus in which the mix (dough) to be extruded is forced through a matrix. The hole diameter of the matrix determines the particle diameter and is generally in the range from 0.3 to 2 mm, and in particular in the range from 0.4 to 1.0 mm. Suitable extruders are, for example, dome extruders or basket extruders which, inter alia, are marketed by companies such as Fitzpatrick or Bepex. For correct consistency of the mix to be granulated, in this case only a low temperature increase results on passing through the matrix (up to approximately 20° C.). Preferably, the extrusion proceeds under temperature control, that is the temperature of the dough should not exceed a temperature of 70° C., in particular 60° C., during extrusion. In particular, the temperature of the dough during extrusion is in the range from 20 to 50° C.

The extruded dough strands leaving the extruder break up into short granule-like particles or can be broken if appropriate using suitable cutting apparatuses. The resultant granule particles typically have a homogeneous particle size, that is a narrow particle size distribution.

In this manner raw granules are obtained having a comparatively high water content which is generally greater than 15% by weight, for example in the range from 15 to 50% by weight, in particular in the range from 20 to 45% by weight, based on the total weight of the moist raw granules. According to the invention, therefore, before coating, drying is carried out in such a manner that the water content of the raw granules is no greater than 15% by weight and preferably is in the range from 1 to 12% by weight, in particular in the range from 3 to 10% by weight, and especially in the range from 5 to 9% by weight.

Generally, final processing of the raw granules will then be carried out. The final processing therefore generally comprises a drying step. This preferably proceeds in a fluidized-bed dryer. In this case, a heated gas, generally air or a nitrogen gas stream, is passed from below through the product layer. The gas rate is customarily set so that the particles are fluidized and swirl. As a result of the gas/particles heat transfer, the water evaporates. Since enzyme-comprising raw granules are generally heat-labile, it is necessary to ensure that the temperature of the raw granules does not rise too high, that is generally not above 80° C., and preferably not above 70° C. In particular, the temperature of the granules during drying is in the range from 30 to 70° C. The drying temperature can be controlled in a simple manner via the temperature of the gas stream. The temperature of the gas stream is typically in the range from 140 to 40° C., and in particular in the range from 120 to 60° C. Drying can proceed continuously or batchwise.

After drying, the granules can be further fractionated by means of a sieve (optional). Coarse material and fines can be ground and returned to the mixer for pasting the granulation mix.

In addition, it has proved to be advantageous to round, that is to say spheronize, the still-moist raw granules before carrying out drying.I In this case, in particular, the formation of unwanted dust fractions in the end product is decreased.

Apparatuses suitable for rounding the moist raw granules are what are termed spheronizers which essentially have a horizontally rotating disk on which the small extruded rods are forced to the wall by the centrifugal force. The small extruded rods break up on the micronotches prefixed by the extrusion process, so that cylindrical particles are formed having a ratio of diameter to length of about 1:1.3 to 1:3. As a result of the mechanical load in the spheronizer, the initially cylindrical particles are somewhat rounded.

According to a further embodiment, the raw granules are produced by spray drying, spray granulation, spray agglomeration, compacting, granulation in a high-shear mixer or similar apparatuses and methods in which mechanical energy is introduced in the form of agitated parts and/or the introduction of a gas stream and thus particle buildup or raw granule production is performed.

A further alternative is raw granule production by absorption. Here, the enzyme concentrate or an enzyme solution is brought into contact with a carrier material (by adding or spraying on the solution). Carrier materials which come into consideration are the carrier materials mentioned above. The enzyme diffuses together with the solvent present in the solution, preferably water, partially or completely into the carrier material in this method. The solvent, preferably water, can subsequently or in parallel be removed by thermal methods. This can be performed, for example, in a fluidized bed, a fluidized-bed dryer or other dryers.

Subsequently, the resultant raw granules can be coated. For this, in a manner known per se, one of the aforesaid coating materials is applied to the raw granules. The coating-forming material can be applied in a manner known per se by application of a solution, dispersion or suspension of the coating-forming material in a suitable solvent, for example water, or by application of a melt of the material. The application of a melt is preferred according to the invention, because the subsequent removal of solvent or dispersion medium can thereby be avoided. This means that for application of a melt, the use of an expensive dryer/coater (for example a fluidized-bed dryer) is not required, but the use of a mixer is possible. Coating with a melt of the material is also termed hereinafter melt coating.

Suitable methods for applying the coating comprise coating in a fluidized bed, and also coating in a mixer (continuously or batchwise), for example in a granulation drum, a ploughshare mixer, for example from Lödige, a paddle mixer, for example from Forberg, a Nauta mixer, a granulating mixer, a granulating dryer, a vacuum coater, for example from Forberg, or a high-shear granulator.

In particular, the raw granules are coated
i) in a fluidized bed, for example by spraying the raw granules with a melt, a solution or dispersion of the material forming the coating; and also
ii) in one of the abovementioned mixing apparatuses by introduction of the raw granules into a melt of the material forming the coating or by spraying or dousing the raw granules with a melt, a solution or dispersion of the material forming the coating.

Coating the raw granules by spraying with a melt, a solution or dispersion in a fluidized bed is particularly preferred according to the invention. Spraying the raw granules with a melt, a solution or dispersion of the material can be carried out in the fluidized-bed apparatus in principle in the bottom-spray method (nozzle is seated in the gas-distribution plate and sprays upwards) or in the top-spray method (coating is sprayed into the fluidized bed from the top).

The raw granules can be coated in the context of the inventive method continuously or batchwise.

According to a first preferred embodiment of the inventive method, the raw granules are charged into a fluidized bed, swirled and, by spraying on an aqueous or nonaqueous, preferably aqueous, dispersion of the material forming the coating, are coated with this material. For this use is made of preferably a liquid which is as highly concentrated as possible and still sprayable, such as, for example, a 10 to 50% strength by weight aqueous dispersion or nonaqueous solution or dispersion of the material.

The solution or dispersion of the material is preferably sprayed on in such a manner that the raw granules are charged into a fluidized-bed apparatus or a mixer and sprayed onto the spray material with simultaneous heating of the charge. The energy is supplied in the fluidized-bed apparatus by contact with heated drying gas, frequently air. Preheating the solution or dispersion can be expedient when as a result spray material having a higher dry substance fraction can be sprayed. When use is made of organic liquid phases, solvent recovery is expedient and the use of nitrogen as drying gas to avoid explosive gas mixtures is preferred. The product temperature during coating should be in the range from about 30 to 80° C., and in particular in the range from 35 to 70° C., and especially in the range from 40 to 60° C. Coating can be carried out in the fluidized-bed apparatus in principle in the bottom-spray method (nozzle is seated in the gas-distribution plate and sprays upwards) or in the top-spray method (coating is sprayed into the fluidized bed from the top). When a mixer is used for coating, after the solution or dispersion is sprayed on, the solvent or the liquid of the dispersion must be removed. This can be carried out in a dryer.

According to a second, particularly preferred embodiment of the inventive method, the raw granules charged into a fluidized bed or mixer are coated with a melt of the material forming the coating. Melt coating in a fluidized bed is preferably carried out in such a manner that the raw granules to be coated are charged into the fluidized-bed apparatus. The material intended for the coating is melted in an external reservoir and pumped, for example via a heatable line to the spraying nozzle. Heating the nozzle gas is expedient. Spraying rate and inlet temperature of the melt are preferably set in such a manner that the material still runs readily on the surface of the granules and evenly coats them. Preheating the granules before spraying the melt is possible. In the case of materials having a high melting point, generally the temperature will be selected in such a manner that a loss of enzyme activity is substantially avoided. The product temperature should therefore preferably be in the range from about 30 to 80° C., and in particular in the range from 35 to 70° C., and especially in the range from 40 to 60° C. Melt coating can also be carried out in principle by the bottom-spray method or by the top-spray method.

Melt coating can be carried out in a mixer in two different ways. Either the granules to be coated are charged into a suitable mixer and a melt of the material is sprayed or poured into the mixer. Another possibility is to mix the hydrophobic material present in solid form with the product. By supplying energy via the vessel wall or via the mixing tools, the hydrophobic material is melted and thus coats the raw granules. According to requirement, from time to time a little release agent can be added. Suitable release agents are, for example, silicic acid, talcum, stearates and tricalcium phosphate, or salts such as magnesium sulfate, sodium sulfate or calcium carbonate.

The solutions, dispersions or melts used for coating can, if appropriate, be admixed with other additives, such as, for example, microcrystalline cellulose, talcum and kaolin, or salts.

In a particular inventive embodiment of the method, the addition of release agents during application of the material or the addition of release agents to the solution, dispersion or melt to be applied can be omitted. This is possible, in particular, when the enzyme cores used have median particle sizes of at least 300 μm, preferably at least 350 μm, in particular at least 400 μm, for example in the range from 300 to 800 μm, preferably in the range from 350 μm to 750 μm, and in particular in the range from 400 μm to 700 μm, and simultaneously the amount of coating material used based on the total particle is no greater than 30% by weight, preferably no greater than 25% by weight, in particular no greater than 20% by weight, and especially no greater than 17% by weight. In these cases, enzyme cores may be coated particularly readily without agglomeration of the particles.

The addition of a flow aid after the coating step can enhance the flow properties of the product. Typical flow aids are silicic acids, for example the Sipernat products from Degussa or the Tixosil products from Rhodia, talcum, stearates and tricalcium phosphate, or salts such as magnesium sulfate, sodium sulfate or calcium carbonate. The flow aids are added to the coated product in an amount of from 0.005% by weight to 5% by weight based on the total weight of the product. Preferred contents are 0.1% by weight to 3% by weight, and particularly preferred 0.2% by weight to 1.5% by weight.

In a further aspect of the invention, the coated or uncoated enzyme granules are mixed with an inert carrier material in order to produce a feed additive which has a lower enzyme activity than the enzyme granules themselves. Preferred carrier materials are the abovementioned carrier materials, or organic substances such as, for example, brans, semolina, coarse break flour or flours of rye, potatoes, wheat, corn or other renewable raw materials, preferably raw materials which are customarily used in animal feeds.

The invention further relates to feed compositions, in particular pelleted feed compositions which, in addition to customary components, comprise at least one feed additive in accordance with the above definition as admixture.

Finally, the invention also relates to the use of a feed additive according to the above definition for producing feed compositions, in particular hydrothermally treated, and especially pelleted, feed compositions.

For production of the feed compositions, the coated or uncoated enzyme granules produced according to the invention are mixed with conventional animal feed (such as, for example, pig-fattening feed, piglet feed, sow feed, broiler feed and turkey feed). The enzyme granule fraction is selected in such a way that the enzyme content is, for example, in the range from 10 to 1000 ppm. Subsequently, the feed is pelleted using a suitable pellet press. For this the feed mixture is customarily conditioned by steam introduction and subsequently pressed through a matrix. Depending on the matrix, pellets of about 2 to 8 mm in diameter can be produced in this way. The highest process temperature occurs in this case during conditioning or during pressing of the mixture through the matrix. Here, temperatures in the range from about 60 to 100° C. can be reached.

The following examples serve to illustrate the invention.

### EXAMPLE 1

a) In an aqueous phytase concentrate having a dry mass content of about 25 to 35% by weight, a pH in the range of 3.7-3.9 and an activity of 26 000 to 36 000 FTU/g, 1% by weight of zinc sulfate hexahydrate, based on the concentrate, was dissolved at 4-10° C.
b) In a mixer having a chopper blade, 700 g of corn starch and 52 g of microcrystalline cellulose were charged, homogenized and to this were added slowly at temperatures of 10 to 30° C. with homogenization simultaneously 630 g of the zinc sulfate-comprising phytase concentrate and 20 g of water. The mixture was homogenized with cooling of the mixer for a further 5 min at temperatures in the range from 10 to 50° C., then the resultant dough was transferred to a dome extruder and the dough was extruded at temperatures in the range from 30 to 50° C. through a matrix having an orifice diameter of 0.7 mm to give 5 cm long strands.
c) The resultant extrudate was rounded in a rounding machine (type P50, from Glatt) for 5 min. at 350 min⁻¹ (speed of rotation of the rotating disk) and then dried to a residual moisture of about 6% by weight in a fluidized-bed dryer (laboratory fluidized bed Aeromat type MP-1 from Niro-Aeromatic) at a temperature of up to 40° C. (product temperature).

The resultant granules had an activity of approximately 18 700 FTU/g. The granules had a particle size of a maximum of 1300 μm and median particle size of 580 μm (sieve analysis).

A product was obtained having the following composition:

Composition:

| | | |
|---|---|---|
| Corn starch | 62.4% | by weight |
| Phytase (dry mass) | 26% | by weight |
| Microcrystalline cellulose: | 5% | by weight |
| Zinc sulfate (ZnSO₄): | 0.6% | by weight |
| Residual moisture: | 6% | by weight |

### EXAMPLE 2

A fines fraction was obtained by means of a vibrating sieve (Retsch) from the enzyme granules of example 1. For this, 200 g of the enzyme granules were placed on a sieve of mesh width 425 μm and sieved for 15 min. A fines fraction (<425 μm) of 38 g was obtained. The granules of the fines fraction had a median particle size of approximately 410 μm (determined by means of Mastersizer S). The activity of the fines fraction was 18 800 FTU/g

### EXAMPLE 3

A coarse particle fraction was obtained by means of a vibrating sieve (Retsch) from the enzyme granules of example 1. For this, 200 g of the enzyme granules were placed on a sieve of mesh width 850 and 600 μm and sieved for 10 min. An intermediate fraction (>600 μm and <850 μm) of 28 g was obtained. The granules of this coarse intermediate fraction had a median particle size of 750 μm (determined by means of sieve analysis). The activity of the coarse fraction was 18 800 FTU/g.

### Experiment 1

### Determination of Metering Accuracy

To assess the metering accuracy of the enzyme granules described under examples 2 and 3, a mixing experiment with subsequent determination of the enzyme activity was carried out as described below. For this, 1 kg of a feed was charged into a Lödige mixer and homogenized. As feed, use was made of a commercially conventional broiler feed having the following composition:

| | |
|---|---|
| Corn | 53.79% |
| HP soybean meal | 36.00% |
| Soybean oil | 6.00% |
| DL-Methionine | 0.31% |
| L-Lysine | 0.10% |
| Threonine | 0.05% |
| Salt lick | 0.38% |
| Feed lime | 1.32% |
| Choline chloride 50% | 0.10% |
| Bolifor MCP-F | 1.45% |
| Vitamin/trace element premix | 0.50% |

In each case by addition of the enzyme granules from examples 2 and 3, a calculated target activity in the feed of 500 FTU/kg was then set. For this, on the basis of the analyzed activity of the enzyme granules, in each case 0.0267 g of the granules from example 2 or example 3 had to be incorporated. Mixing was performed for 10 min in each case. After mixing, from each batch in each case 10 samples each of 50 g were withdrawn and their enzyme activity was determined. In the table below, the results are summarized:

| | Granules | |
|---|---|---|
| | Granules | Granules |
| | from ex. 2 | from ex. 3 |
| Median particle diameter [μm] | 410 | 750 |
| Granule activity [FTU/g] | 18 800 | 18 800 |
| Amount of granules weighed out per | 0.0267 | 0.0267 |
| 1 kg of feed [g] | | |
| Target activity in the feed [FTU/kg] | 500 | 500 |
| Activity in the feed, sample 1 [FTU/kg] | 755 | 604 |
| Activity in the feed, sample 2 [FTU/kg] | 475 | 886 |
| Activity in the feed, sample 3 [FTU/kg] | 585 | 589 |
| Activity in the feed, sample 4 [FTU/kg] | 822 | 279 |
| Activity in the feed, sample 5 [FTU/kg] | 493 | 1126 |
| Activity in the feed, sample 6 [FTU/kg] | 544 | 729 |
| Activity in the feed, sample 7 [FTU/kg] | 289 | 507 |
| Activity in the feed, sample 8 [FTU/kg] | 582 | 509 |
| Activity in the feed, sample 9 [FTU/kg] | 587 | 1107 |
| Activity in the feed, sample 10 [FTU/kg] | 530 | 683 |
| Mean activity in the feed [FTU/kg] | 566 | 702 |
| Standard deviation of activity in the | 147 | 270 |
| feed [FTU/kg] | | |

The standard deviation of the activity in the feed is greater with the coarser granules (ex. 3) than with the granules from example 2.

1. A phytase-comprising enzyme granule for feeds, comprising one or more particles which have a weight-average particle size in the range from 300 to 800 μm and a specific phytase activity which, expressed in FTU/g, is at least 13 000 and does not exceed a value of
FTUₘₐₓ=6000[FTU g⁻¹]·D⁻³·mm³,
or a specific phytase activity which, expressed in FTU/g, is at least 7000, and does not exceed a value of
FTUₘₐₓ=2000[FTU g⁻¹]·D⁻³·mm³,
D being the weight-average particle diameter of the granule particles in mm.

2. The enzyme granule according to claim 1, wherein 90% by weight of the particles have a particle diameter of greater than 100 μm (D₁₀ value).

3. The enzyme granule according to claim 1, wherein 90% by weight of the particles have a particle diameter of no greater than 1200 μm (D₉₀ value).

4. The enzyme granule according to claim 1, wherein the particle size distribution of the granule particles being characterized has a D₉₀/D₁₀ value <3.5.

5. The enzyme granule according to claim 1, wherein the granule particles have a roundness factor less than 2.

6. The enzyme granule according to claim 1 comprising, in addition to the phytase, at least one solid carrier material suitable for feeds.

7. The enzyme granule according to claim 1, wherein the particles of the granule comprise
A) a core which comprises at least one phytase and at least one solid carrier material suitable for feeds, and
b) a coating arranged on the core.

8. The enzyme granule according to claim 7, wherein at least 70% by weight of the at least one coating arranged on the surface of the core comprises substances selected from waxes, saturated fatty acids, the esters of saturated fatty acids, polyolefins, cellulose derivatives, polyvinyl acetate, polyvinylpyrrolidone, polyvinyl alcohol, vinyl acetate/vinylpyrrolidone copolymers, polyalkylene glycols or mixtures thereof.

9. The enzyme granule according to claim 7, wherein at least 90% by weight of the at least one coating arranged on the surface of the core comprises water-insoluble hydrophobic substances.

10. The enzyme granule according to claim 7, wherein the weight ratio of core to coating is in the range from 70:30 to 99:1.

11. The enzyme granule according to claim 6, wherein the carrier material comprises at least one water-insoluble polymeric carbohydrate.

12. The enzyme granule according to claim 1, comprising, in addition, a water-soluble binder.

13. The enzyme granule according to claim 12, wherein the water-soluble binder is selected from polyvinyl alcohol or water-soluble polysaccharides.

14. The enzyme granule according to claim 13, wherein the water-soluble polymeric binder comprises a neutral cellulose ether.

15. The enzyme granule according to claim 1, additionally comprises a salt stabilizing the enzyme.

16. The enzyme granule according to claim 15, wherein the salt is selected from zinc sulfate or magnesium sulfate.

17. A method of producing feed comprising utilizing the phytase-comprising enzyme granule according to claim 1 in feeds.

18. A feed comprising at least one type of phytase-comprising enzyme granule according to claim 1 and customary feed components.

19. The feed according to claim 18 in the form of a pelleted feed.

20. The enzyme granule of claim 14, wherein the neutral cellulose ether is methylcellulose.
